(19) United States
(12) Patent Application Publication
(10) Pub. No.: US 2005/0176938 A1
(43) Pub. Date: Aug. 11, 2005

(54) Anhydrate/hydrate of an erythromycin derivative and processes for preparing said anhydrate/hydrate

(75) Inventors: Kenji Kamada, Tokyo (JP); Akira Hiraide, Tokyo (JP); Kaichiro Koyama, Tokyo (JP); Hitoshi Shimizu, Tokyo (JP); Gary H. Ward, San Diego, CA (US); Vandana K. Cheruvallath, San Diego, CA (US); Stephan Parent, Lafayette, IN (US)

Correspondence Address: BROWDY AND NEIMARK, P.L.L.C.;624 NINTH STREET, NW, SUITE 300, WASHINGTON, DC, 20001-5303, US

(73) Assignee: Chugai Seiyaku Kabushiki Kaisha, Tokyo (JP)

(21) Appl. No.: 10/501,215

(22) PCT Filed: Jan. 14, 2003
(86) PCT No.: PCT/JP03/00203

(30) Foreign Application Priority Data
Jan. 11, 2002 (JP) 2002-004680
Mar. 19, 2003 (JP) 2002-076708

(51) Int. Cl. C07H 17/08

(52) U.S. Cl. 536/7.1

(57) ABSTRACT

Crystal form F of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)-ene-3,7-dione (E)-2-butenedioic acid salt (2:1) showing strong X-ray diffraction peaks at diffraction angles 2θ=6.6° and 8.5°, Crystal form D anhydrate of said compound showing strong X-ray diffraction peaks at diffraction angles 2θ=7.1°, 13.5° and 14.2°, Crystal form D X-hydrate of said compound showing strong X-ray diffraction peaks at diffraction angles 2θ=7.1° and 14.2° but not showing a strong X-ray diffraction peak at a diffraction angle 2θ=13.5°, Solvate Crystal forms G1, G2 and G3 of said compound characterized by 2-theta angle positions in the powder X-ray diffraction pattern of 5.4°, 10.4°, 10.7° and 12.1°. Crystal form D X-hydrate of said compound has more preferred properties for use as a pharmaceutical material such as higher stability as compared with Crystal form D anhydrate of said compound.

### TECHNICAL FIELD

The present invention relates to a novel hemifumarate crystal, anhydrate and X-hydrate of an erythromycin derivative as well as processes for preparing said anhydrate and X-hydrate, which are useful as pharmaceutical and therapeutic agents.

### BACKGROUND ART

The compound of formula (I): 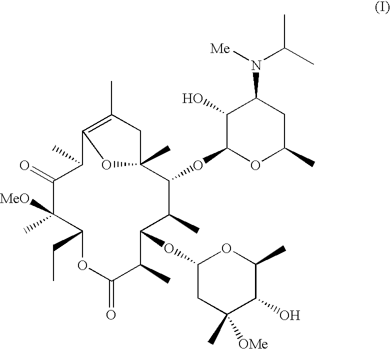
(N-demethyl-N-isopropyl-12-methoxy-11-oxo-8,9-anhydroerythromycin A-6,9-hemiacetal) is described in JPA (Japanese Patent Publication for Laying-open) 1994-56873 (WO93/24509), JPA 1997-100291 (WO97/06177), etc., and known to have the effect of promoting gastrointestinal motility.

Processes for preparing this compound are described in JPA 1997-100291, Bioorg. & Med. Chem. Lett. Vol. 4, No. 11, p. 1347, 1994, etc.

Fumarate crystals of the compound of formula (I) are known in the art and have been designated Crystal form A, Crystal form C, and Crystal form D and can be obtained by the processes described in JPA 1997-100291.

Crystal form A can be obtained by recrystallizing a fumarate of the compound of formula (I) in an alcoholic solvent such as a mixed solvent of methanol and isopropanol in a molar ratio of the compound of formula (I) to the fumarate of 2:1.

Crystal form C can be obtained by treating a fumarate of the compound of formula (I) with ethyl acetate in a molar ratio of the compound of formula (I) to the fumarate of 1:1.

Crystal form D can be obtained by treating a fumarate of the compound of formula (I) with a mixed solvent of ethyl acetate and water in a molar ratio of the compound of formula (I) to the fumarate of 2:1.

JPA 1997-100291 describes that Crystal form D has excellent properties as a pharmaceutical or pharmaceutical material such as excellent stability as compared with crystal forms A, C and D.

However, no report has shown the presence of the anhydrate and hydrate of crystal form D of the compound of formula (I).

### DISCLOSURE OF INVENTION

As a result of careful studies on crystal forms of the compound described above, we found a fumarate crystal of the compound of formula (I) having a novel crystal form and also found the presence of a hydrate and an anhydrate of crystal form D obtained by treating said novel crystal, and particularly that said hydrate has more preferred properties as a pharmaceutical material, whereby we accomplished the present invention on the basis of these findings.

Accordingly, the present invention relates to a hemifumarate crystal of a compound of formula (I): 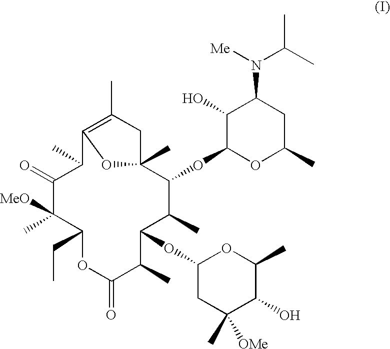
characterized by 2-theta angle positions in the powder X-ray diffraction pattern of 6.6° and 8.5° as measured by X-ray diffractometry using Cu—Kα radiation (hereinafter referred to as crystal form F).

The present invention also relates to a hemifumarate anhydrate of the compound of formula (I) above characterized by 2-theta angle positions in the powder X-ray diffraction pattern of 7.1°, 13.5° and 14.2° as measured by X-ray diffractometry using Cu—Kα radiation (hereinafter referred to as Crystal form D anhydrate).

The present invention also relates to a hemifumarate X-hydrate of the compound of formula (I) above characterized by 2-theta angle positions in the powder X-ray diffraction pattern of 7.1° and 14.2° but not showing a strong X-ray diffraction peak at a diffraction angle 2θ=13.5° as measured by X-ray diffractometry using Cu—Kα radiation (hereinafter referred to as Crystal form D X-hydrate).

The present invention also relates to a process for preparing Crystal form D X-hydrate comprising conditioning Crystal form D anhydrate by methods known in the art, such as by storing it in a humidifying room or spraying it with humidifying steam.

The present invention also relates to a process for preparing Crystal form D anhydrate comprising obtaining it via Crystal form F.

The present invention also relates to a process for preparing Crystal form D X-hydrate comprising obtaining it through Crystal form F.

The present invention also relates to a process for preparing Crystal form D X-hydrate comprising conditioning Crystal form D anhydrate obtained through Crystal form F.

The present invention relates to a hemifumarate crystal of a compound of formula (I) characterized by 2-theta angle positions in the powder X-ray diffraction pattern of 5.4°, 10.4°, 10.7° and 12.1°.

The present invention also relates to a hemifumarate crystal of a compound of formula (I) above containing acetone and showing strong X-ray diffraction peaks at diffraction angles 2 theta=5.4°, 10.4°, 10.7° and 12.1° measured by X-ray diffractometry using Cu—Kα radiation.

The present invention also relates to a hemifumarate crystal of a compound of formula (I) above containing methylethylketone and showing strong X-ray diffraction peaks at diffraction angles 2 theta=5.4°, 10.4°, 10.7° and 12.1° measured by X-ray diffractometry using Cu—Kα radiation.

The present invention also relates to a hemifumarate crystal of a compound of formula (I) above containing tetrahydrofuran and showing strong X-ray diffraction peaks at diffraction angles 2 theta=5.4°, 10.4°, 10.7° and 12.1° measured by X-ray diffractometry using Cu—Kα radiation.

The present invention also relates to a process for preparing a hemifumarate X-hydrate of a compound of formula (I) above showing strong X-ray diffraction peaks at diffraction angles 2 theta=5.4°, 10.4°, 10.7° and 12.1° measured by X-ray diffractometry using Cu—Kα radiation, said process comprising the step of treating a hemifumarate crystal of the compound of formula (I) characterized by 2-theta angle positions in the powder X-ray diffraction pattern of 5.4°, 10.4°, 10.7° and 12.1°, to obtain said hydrate.

The present invention also relates to a process for preparing a hemifumarate X-hydrate of a compound of formula (I) above containing acetone and showing strong X-ray diffraction peaks at diffraction angles 2 theta=5.4°, 10.4°, 10.7° and 12.1° measured by X-ray diffractometry using Cu—Kα radiation, said process comprising the step of treating a hemifumarate crystal of the compound of formula (I) above characterized by 2-theta angle positions in the powder X-ray diffraction pattern of 5.4°, 10.4°, 10.7° and 12.1°, to obtain said hydrate.

The present invention also relates to a process for preparing a hemifumarate X-hydrate of a compound of formula (I) above containing methylethylketone and showing strong X-ray diffraction peaks at diffraction angles 2 theta=5.4°, 10.4°, 10.7° and 12.1° measured by X-ray diffractometry using Cu—Kα radiation, said process comprising the step of treating a hemifumarate crystal of the compound of formula (I) characterized by 2-theta angle positions in the powder X-ray diffraction pattern of 5.4°, 10.4°, 10.7° and 12.1°, to obtain said hydrate.

The present invention also relates to a process for preparing a hemifumarate X-hydrate of a compound of formula (I) above containing tetrahydrofuran and showing strong X-ray diffraction peaks at diffraction angles 2 theta=5.4°, 10.4°, 10.7° and 12.1° measured by X-ray diffractometry using Cu—Kα radiation, said process comprising the step of treating a hemifumarate crystal of the compound of formula (I) characterized by 2-theta angle positions in the powder X-ray diffraction pattern of 5.4°, 10.4°, 10.7° and 12.1°, to obtain said hydrate.

The present invention also relates to a process for preparing a hemifumarate anhydrate of a compound of formula (I) above characterized by 2-theta angle positions in the powder X-ray diffraction pattern of 7.1°, 13.5° and 14.2°, said process comprising the step of obtaining said anhydrate by treating a hemifumarate crystal of Crystal form G, G1, G2 or G3.

The present invention also relates to a process for preparing a hemifumarate X-hydrate of a compound of formula (I) above characterized by 2-theta angle positions in the powder X-ray diffraction pattern of showing strong X-ray diffraction peaks at diffraction angles 2θ=7.1° and 14.2°, said process comprising the step of obtaining said hydrate by treating a hemifumarate crystal of Crystal form G, G1, G2 or G3.

The present invention also relates to a process for preparing a hemifumarate X-hydrate of a compound of formula (I) above characterized by 2-theta angle positions in the powder X-ray diffraction pattern of 7.1° and 14.2°, said process comprising the step of treating a hemifumarate anhydrate of the compound of formula (I) characterized by 2-theta angle positions in the powder X-ray diffraction pattern of 7.1°, 13.5° and 14.2°, wherein said anhydrate is obtained by treating a hemifumarate crystal of Crystal form G, G1, G2 or G3.

### BRIEF DESCRIPTION OF DRAWINGS

FIG. 1 shows an example of a powder X-ray diffraction pattern of Crystal form F.

FIG. 2 shows an example of a powder X-ray diffraction pattern of Crystal form D anhydrate.

FIG. 3 shows an example of a powder X-ray diffraction pattern of Crystal form D X-hydrate.

FIG. 4 shows an example of measured results of moisture absorption isotherms of Crystal form D anhydrate and Crystal form D X-hydrate.

FIG. 5 shows a representative XRPD pattern of Crystal form G1.

FIG. 6 shows a representative XRPD pattern of Crystal form G2.

FIG. 7 shows a representative XRPD pattern of Crystal form G3.

FIG. 8 shows DSC and TGA curves of Crystal form G1.

FIG. 9 shows a TGA desolvation curve of Crystal form G1.

FIG. 10 shows a TGA desolvation curve of Crystal form G2.

FIG. 11 shows IR spectra of Crystal form G2 volatiles.

FIG. 12 shows a TGA desolvation curve of Crystal form G3.

FIG. 13 shows IR spectra of Crystal form G3 volatiles.

### BEST MODE FOR CARRYING OUT THE INVENTION

Crystal form F of the present invention is characterized by the diffraction pattern as shown in FIG. 1 as measured by X-ray diffractometry using Cu—Kα radiation. As shown in FIG. 1, it is characterized by 2-theta angle positions in the powder X-ray diffraction pattern of 6.6° and 8.5°. More specifically, it is characterized by 2-theta angle positions in the powder X-ray diffraction pattern of 6.6°, 8.5°, 16.6°, 20.8° and 23.5°.

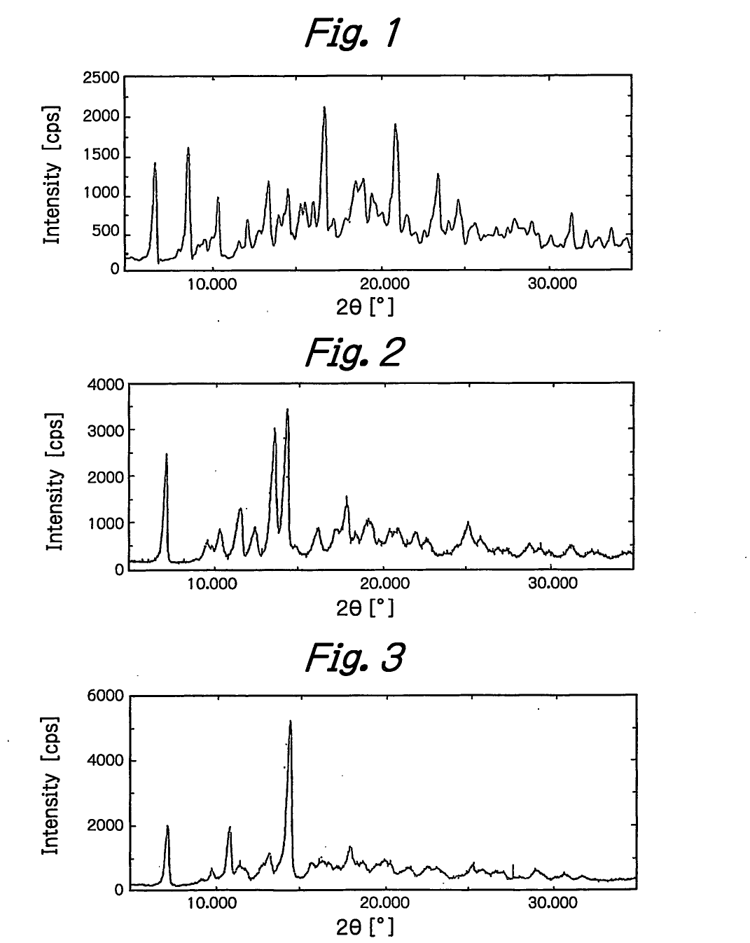

Crystal form D anhydrate of the present invention is characterized by 2-theta angle positions in the powder X-ray diffraction pattern as shown in FIG. 2 as measured by X-ray diffractometry using Cu—Kα radiation. As shown in FIG. 2, Crystal Form D anhydrate is characterized by 2-theta angle positions in the powder X-ray diffraction pattern of 7.1°, 13.5° and 14.2°. More specifically, it is characterized by 2-theta angle positions in the powder X-ray diffraction pattern of 7.1°, 9.4°, 10.2°, 12.3°, 13.5°, 14.2° and 16.1°. Among such chracteristic angle positions, the angle position of 13.5° is a characteristic angle position that is not found in crystal form D X-hydrate.

Crystal form D X-hydrate of the present invention is characterized by 2-theta angle positions in the powder X-ray diffraction pattern as shown in FIG. 3 as measured by X-ray diffractometry using Cu—Kα radiation, wherein X in X-hydrate is at about ½.

As shown in FIG. 3, Crystal form D hydrate is characterized by 2-theta angle positions in the powder X-ray diffraction pattern of 7.1° and 14.2°, but does not show a X-ray diffraction peak at a diffraction angle 2θ=13.5° (the strong peak at a diffraction angle 2θ=13.5° found in Crystal form D anhydrate is not present). More specifically, it is characterized by 2-theta angle positions in the powder X-ray diffraction pattern of 7.1°, 10.7°, 14.2°, 15.7° and 16.7°.

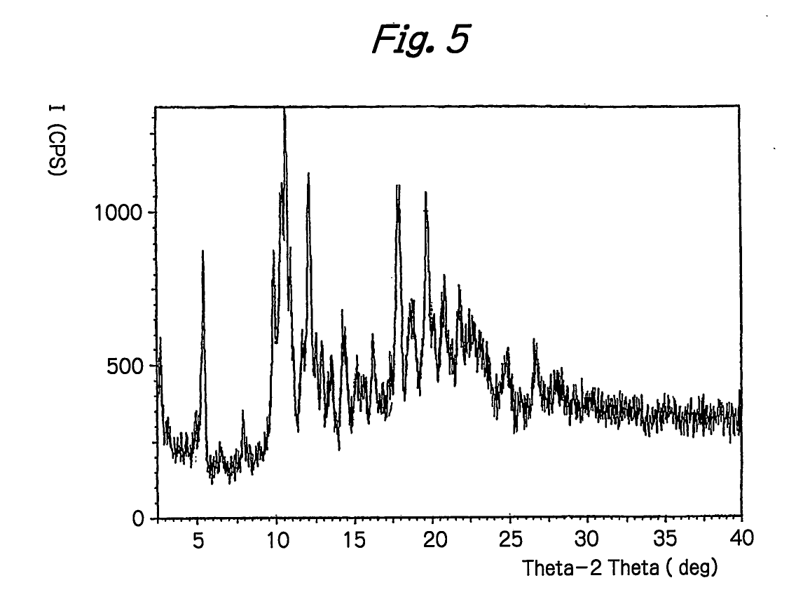

Crystal form G1 of the present invention is characterized by 2-theta angle positions in the powder X-ray diffraction pattern as shown in FIG. 5 as measured by X-ray diffractometry using Cu—Kα radiation. As shown in FIG. 5, the crystal form G1 is characterized by 2-theta angle positions in the powder X-ray diffraction pattern of 5.4°, 10.4°, 10.7° and 12.1°. More specifically, it is characterized by containing acetone and having 2-theta angle positions in the powder X-ray diffraction pattern of 5.4°, 10.4°, 10.7° and 12.1°.

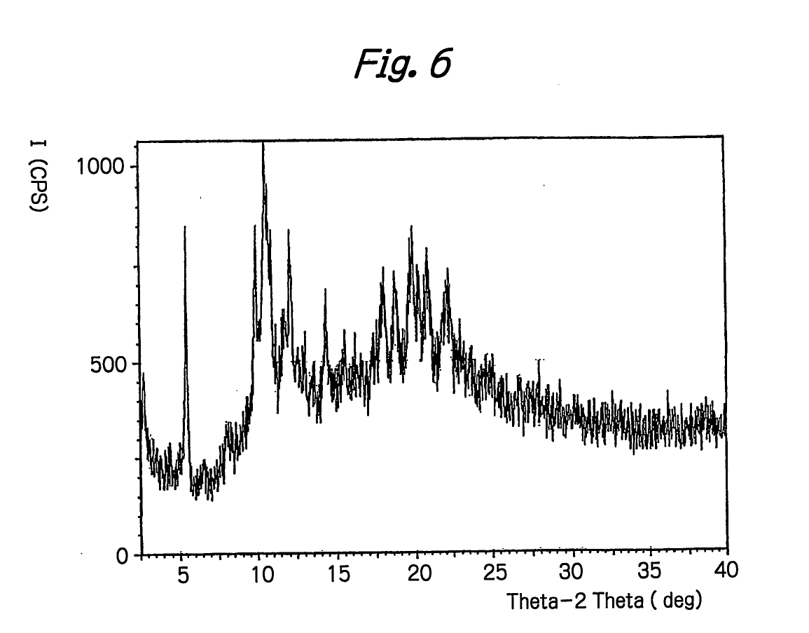

Crystal form G2 of the present invention is characterized by 2-theta angle positions in the powder X-ray diffraction pattern as shown in FIG. 6 as measured by X-ray diffractometry using Cu—Kα radiation. As shown in FIG. 6, the crystal form G2 is characterized by 2-theta angle positions in the powder X-ray diffraction pattern of 5.4°, 10.4°, 10.7° and 12.1°. More specifically, it is characterized by containing methylethylketone and having 2-theta angle positions in the powder X-ray diffraction pattern of 5.4°, 10.4°, 10.7° and 12.1°.

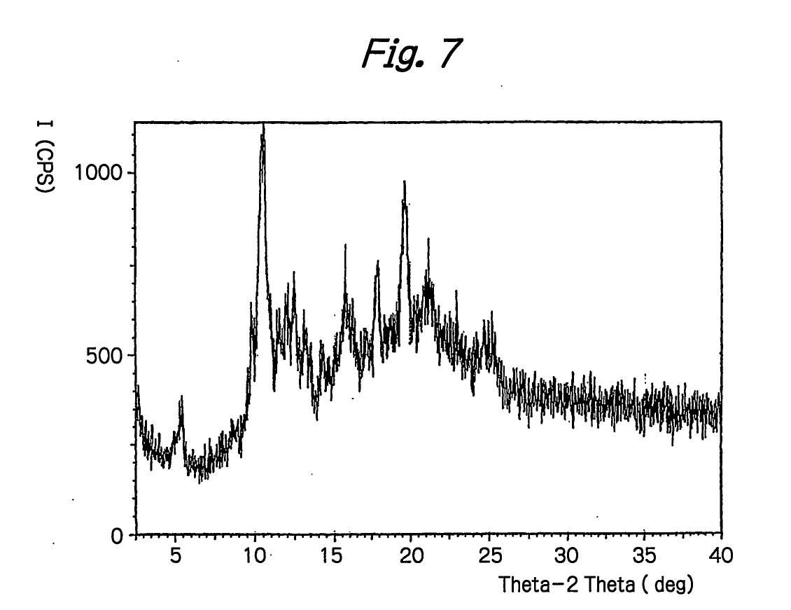

Crystal form G3 of the present invention is characterized by 2-theta angle positions in the powder X-ray diffraction pattern as shown in FIG. 7 as measured by X-ray diffractometry using Cu—Kα radiation. As shown in FIG. 7, the crystal form G3 is characterized by 2-theta angle positions in the powder X-ray diffraction pattern of 5.4°, 10.4°, 10.7° and 12.1°. More specifically, it is characterized by containing tetrahydrofuran and having 2-theta angle positions in the powder X-ray diffraction pattern of 5.4°, 10.4°, 10.7° and 12.1°.

The X-ray diffraction angles described above can be measured with various commercially available equipments such as powder X-ray diffractometers using Cu—Kα radiation as well as other detection methods known in the art, or the like methods. The principle of powder X-ray diffractometry is described in detail at B614-B619 of the Practical Guide of the 14th revision of Pharmacopoeia of Japan (published by Hirokawa Publishing Co., 2001) or the like, and an error of diffraction angle on the order of ±0.2° is normally permissible.

Next, the present invention is specifically explained.

Crystal form F of the present invention can be prepared from Crystal form E, for example. Crystal form E means a hemifumarate of the compound of formula (I) above characterized by containing tetrahydrofuran and having 2-theta angle positions in the powder X-ray diffraction pattern of 5.6° and 10.4° as measured by X-ray diffractometry using Cu—Kα radiation.

Crystal form E can be obtained by treating Crystal form C at 20-40° C. in a mixed solvent of ethyl acetate and water. Crystal form C here can be used after isolation or preferably in suspension in the solvent For example, Crystal form C is preferably obtained by treating Crystal form A with ethyl acetate and water is added to this suspension of Crystal form C in ethyl acetate.

The ratio of ethyl acetate to water in the mixed solvent of ethyl acetate and water used for suspension is normally from about 99:1 to about 95:5, preferably from about 97:3 to about 95:5. The temperature for suspension is normally from about 20 to about 40° C., preferably from about 20 to about 30° C. At temperatures of less than about 20° C., the tendency is for Crystal form E or a mixture of Crystal forms C and E to transition into Crystal form D. The suspension period is normally from about 30 to about 300 minutes, preferably from about 60 to about 240 minutes.

The resulting Crystal form E is separated from the solvent by filtration, centrifugation, etc. The separated Crystal form E is preferably dried under reduced pressure. The drying temperature is normally from about 20 to about 60 ° C., preferably from about 30 to about 50° C.

Crystal form C can be obtained by treating Crystal form A with ethyl acetate as described in JPA 1997-100241, for example.

Crystal form A can be obtained by treating a fumarate of the compound of formula (I) with a mixed solvent of methanol and isopropanol as described in JPA 1994-56873 or JPA 1997-100241, for example.

Crystal form F of the present invention can be obtained by suspending Crystal form E in a mixed solvent of ethyl acetate and water at less than 20° C. The ratio of ethyl acetate to water in the mixed solvent of ethyl acetate and water used here is preferably from about 98.1:1.9 to about 97:3. The temperature for suspension is from about 10 to about 20° C. (preferably from about 11 to about 19° C., more preferably from about 13 to about 18° C.). In order to promote conversion into the crystal of the present invention or to increase the yield, the suspension may be subsequently cooled to from about −20 to about 10° C. (preferably from about −15 to about 10° C.). The suspension period is normally from about several minutes to about 20 hours, preferably 5 minutes to 4 hours, more preferably from about 10 minutes to about 2 hours. The subsequent suspension step normally lasts for about several minutes to about 20 hours, preferably about 1 hour. Thus obtained Crystal form F of the present invention is separated from the solvent by filtration, centrifugation or the like to give a wet crystal.

The suspension periods of time specifically shown above are minimum periods of time for producing each crystal, and they may be extended depending on the crystal growth rate or the convenience of the production process. When Crystal form F of the present invention is prepared through Crystal form E, Crystal form F can be prepared continuously from Crystal form C through Crystal form E only by controlling the temperature without isolating Crystal form E.

Crystal form F of the present invention can also be obtained either directly or indirectly from the Crystal form D anhydrate or the Crystal form D X-hydrate (described below). The mixed solvent system composed of ethyl acetate and water (used in treating the E-form to obtain the F-form) is also used in obtaining the F-form from the Crystal form D. Crystal form D anhydrate or Crystal form D X-hydrate here is preferably indirectly contacted with a mixed solvent of ethyl acetate and water by placing it in an (preferably saturated) atmosphere containing such mixed solvent, preferably in a saturated atmosphere. The atmosphere here is preferably an inert gas such as air, nitrogen, carbon dioxide or argon.

Crystal form F that is obtained as described above is dried under reduced pressure, for example, to give Crystal form D anhydrate of the present invention. The drying temperature here is preferably from about 20 to about 70° C. Crystal form D anhydrate of the present invention can also be obtained by drying Crystal form D X-hydrate described below. However, this Crystal form D anhydrate must be stored under conditions resisting moisture absorption (i.e., resisting transition into Crystal form D X-hydrate) due to its hygroscopic nature such that it is partially or totally transferred into Crystal form D X-hydrate by adsorbing water in the atmosphere if it remains in normal atmosphere.

Crystal form D anhydrate of the present invention can also be obtained by drying Crystal form G1-, G2-, or G3 under reduced pressure. Thus obtained Crystal form D anhydrate may be conditioned by a known method such as leaving it in a humidifying steam room or spraying it with humidifying steam, to give Crystal form D X-hydrate.

Crystal form D X-hydrate of the present invention can be prepared, for example, by conditioning Crystal form D anhydrate described above by methods known in the art, such as by storing it in a humidifying steam room or spraying it with humidifying steam. Specifically, it can be prepared by conditioning Crystal form D anhydrate using a commercially available apparatus such as an air-circulating dryer or vibro-fluidized bed apparatus, for example. The atmosphere during conditioning is preferably an inert gas such as air, nitrogen, carbon dioxide and argon.

The transition point from Crystal form D anhydrate into Crystal form D X-hydrate is a relative humidity of about 30% RH to about 40% RH at about 25° C., and the transition point from Crystal form D X-hydrate into Crystal form D anhydrate is a relative humidity of about 30% RH to about 20% RH at about 25° C. Either transition readily occurs, specifically within a short period of about 10 minutes or less on a small scale. To ensure transition, it is preferable to maintain Crystal form D anhydrate at a relative humidity of 20% RH or less at 25° C. and Crystal form D X-hydrate at a relative humidity of about 40% RH or more at about 25° C. Each of the transition points tends to shift to low humidity side at temperatures lower than 25° C. and to high humidity side at temperatures higher than 25° C.

The residual solvent (ethyl acetate) level decreases as Crystal form D anhydrate transitions into Crystal form D X-hydrate. Crystal form D X-hydrate is more stable than Crystal form D anhydrate. Moreover, Crystal form D X-hydrate is advantageous for industrial production because it is more easily handled than Crystal form D anhydrate. For these reasons, Crystal form D X-hydrate of the present invention is especially useful as a pharmaceutical material.

Crystal form D anhydrate of the present invention is useful as a material or an intermediate for the synthesis of this Crystal form D X-hydrate.

Crystal form F of the present invention is useful as a material or an intermediate for the synthesis of these Crystal form D anhydrate and D-form—X-hydrate.

A hemifumarate anhydrate of Crystal form D is obtained through a hemifumarate crystal of Crystal form G1, G2 or G3.

The residual solvent level described above can be determined by a known method such as gas chromatography. Gas chromatography is described in detail at B98-B114 of the Practical Guide of the 14th revision of Pharmacopoeia of Japan (published by Hirokawa Publishing Co., 2001) or the like. The measurement error by gas chromatography is normally within about ±1%.

Iso-structural solvate G-form crystals, namely, Crystal form G1, Crystal form G2 and Crystal form G3 of the present invention can be obtained by directly or indirectly contacting hydrate Crystal form D described below with a specific solvent as described below.

Separation for Stereoisomers

Compounds of the present invention may exist as stereoisomers wherein, asymmetric or chiral centers are present These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Separation for Formulation

The present invention provides pharmaceutical compositions which comprise compounds of the present invention formulated together with one or more non-toxic pharmaceutically acceptable carriers. The pharmaceutical compositions can be formulated for oral administration in solid or liquid form, for parenteral injection or for rectal administration. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

Further included within the scope of the present invention are pharmaceutical compositions comprising one or more of the compounds of formula (I-II) prepared and formulated in combination with one or more non-toxic pharmaceutically acceptable compositions. The pharmaceutical compositions can be formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. (United States Pharmacopoeia) and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N.Y., (1976), p 33 et seq.

The terms “pharmaceutically acceptable salts, esters and amides,” as used herein, refer to carboxylate salts, amino acid addition salts, zwitterions, esters and amides of compounds of formula (I-II) which are, within the scope of sound medical judgement, suitable for use in contact with the tissues of humans and lower animals without undue toxicity, irritation, allergic response, and the like, are commensurate with a reasonable benefit/risk ratio, and are effective for their intended use.

The compounds of the present invention can be used in the form of pharmaceutically acceptable salts derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66:1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the invention include phosphate, tris and acetate.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to a parent compound of formula (I-II), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987), hereby incorporated by reference.

Dosage forms for topical administration of a compound of this invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

### EXAMPLES

The following examples further illustrate the present invention without, however, limiting the invention thereto. In the following examples, NMR spectra were measured by using a nuclear magnetic resonance spectrometer JNM-ECP500SS (made by JEOL) and only characteristic peaks were shown. Powder X-ray diffraction spectra were measured by using a powder X-ray diffractometer RINT-1100 (made by Rigaku). Residual solvent levels were measured by using a gas chromatograph GC-17A (made by Shimadzu) within an error of about ±1%. The staring material Dihydroxy compound described below (compound 1) can be prepared according to the process described in JPA 1997-100241 or modifications thereof.

### [Example 1]

### Preparation of Crystal Form F of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)ene-3,7-dione 3)-2-butenedioic acid salt (2:1) (Crystal Form F)

(1) Synthesis of a Z Compound (Compound 2)

To Dihydroxy compound (compound 1) (15 kg) of the formula below: 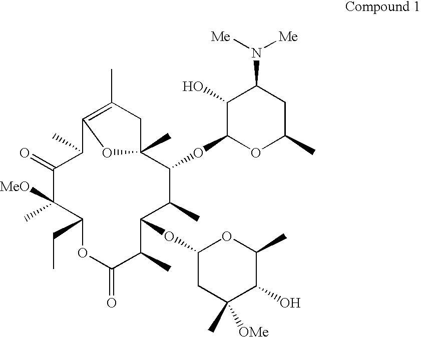
and sodium hydrogencarbonate (12.1 kg) was added ethyl acetate (67.7 kg). This mixed solution was heated to 55° C. and then stirred with benzyloxycarbonyl chloride (7.0 kg) for 1 hour. The mixed solution was further stirred with benzyloxycarbonyl chloride (31.6 kg) for 1 hour and then cooled to 28° C., so that the starting Dihydroxy compound (compound 1) and the reaction intermediate (compound 1 added with a benzyloxycarbonyl group) were completely lost and converted into a Z compound (compound 2) of the formula below:

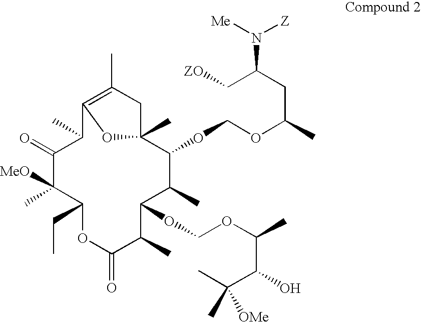

This solution was stirred with pyridine (0.016 kg) for 0.5 hours. The operation of stirring with pyridine (0.016 kg) for 0.5 hours was further repeated twice, and then pyridine (5.6 kg) was added. This solution was stirred with water (120.0 kg) and then separated to remove aqueous phase, and organic phases were washed with saturated brine (75.0 kg) and the combined organic phases were then concentrated under reduced pressure to give an oily Z compound (compound 2).

(2) Synthesis of Monomethyl Compound (Compound 3)

The Z compound (compound 2) obtained in (1) above was stirred with methanol (59.3 kg), 10% palladium-carbon (4.0 kg) and sodium hydrogencarbonate (17.3 kg) for 2 hours at 25-50° C. in a hydrogen atmosphere (0.1 MPa-0.4 MPa) without isolation/purification, so that the starting Z compound (compound 2) and the reaction intermediate (compound 2 having a benzyloxycarbonyl group deprotected) were completely lost and converted into Monomethyl compound (compound 3) of the formula below:

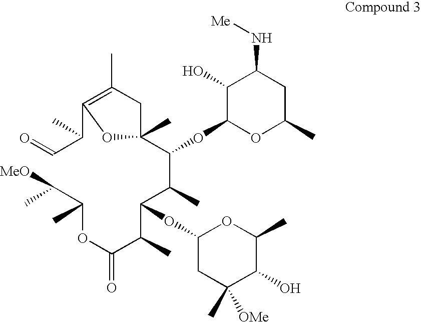

The reaction solution was filtered to remove palladium-carbon and then concentrated under reduced pressure. The residue was dissolved in ethyl acetate (94.7 kg) and the solution was stirred with an aqueous saturated sodium hydrogen carbonate (52.5 kg) and then separated to remove aqueous phase. Then, organic phases were washed with saturated brine (52.5 kg) and the combined organic phases were then concentrated under reduced pressure to give oily Monomethyl compound (compound 3).

(3) Synthesis of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)-ene-3,7-dione

Monomethyl compound (compound 3) obtained in (2) above was dissolved in 1,3-dimethyl-2-imidazolidinone (63.1 kg) without isolation/purification, and the solution was stirred with triethylamine (20.9 kg) and isopropyl iodide (31.5 kg) for 6 hours under heating at 75° C., so that 96% of the starting Monomethyl compound (compound 3) was converted into the title compound. The reaction solution was cooled to 30° C. or less and then stirred with ethyl acetate (88.0 kg), 25% aqueous ammonia (3.8 kg) and water (33.8 kg) and then separated to remove aqueous phase. The operation of stirring with water followed by separation to remove aqueous phase was further repeated twice. The combined organic phases were concentrated under reduced pressure to give the title compound of formula (I) below: 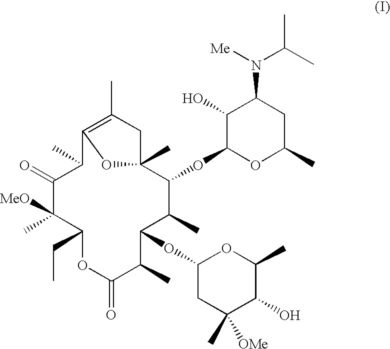
(4) Synthesis of Crystal Form D of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)-ene-3,7-dione (E)-2-butenedioic acid salt (2:1)

N-demethyl-N-isopropyl-12-methoxy-11-oxo-8,9-anhydroerythromycin A-6,9-hemiacetal obtained in (3) above was combined with fumaric acid (1.2 kg) and isopropanol (117.8 kg) without isolation/purification and heated to 70° C. and then cooled to 10° C. or less at 20° C./h. The precipitated crystal was filtered out to give a crystal of the title compound (wet powder; dry yield 83.5%, purity 91.62%). This wet powder was combined with isopropanol (109.9 kg) and heated to 72° C. and then cooled to 10° C. or less at 20° C./h. The precipitated crystal was filtered out to give a crystal of the title compound (wet powder; purity 98.58%).

(5) Purification of Crystal Form D of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)-ene-3,7-dione (E)-2-butenedioic acid salt (2:1).

Crystal form D obtained in (4) above was combined with methanol (2.5 v/w based on the dry weight of the compound obtained in (4) above) and isopropanol (7.5 v/w based on the dry weight of the compound obtained in (4) above) without isolation/purification and heated to 60° C. and then cooled to 0° C. or less at 20° C./h. The precipitated crystal was filtered out to give a crystal of the title compound (wet powder). The resulting crystal was subjected to said operation once again without drying to give a crystal of the title compound (wet powder; purity 99.97%). This wet powder was dried under vacuum for 12 hours to give a crystal of the title compound (purity 99.93%).

(6) Acquisition of Crystal Form F of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)-ene-3,7-dione (E)-2-butenedioic acid salt (2:1) (Crystal Form F)

Crystal form D obtained in (5) above (10.9 kg) was dissolved in ethyl acetate (78.7 kg) and methanol (6.9 kg) and then the solution was concentrated to dryness under reduced pressure. This dry concentrate was stirred with ethyl acetate (81.6 kg) at 25° C. for 2 hours to give Crystal form C. This Crystal form C was combined with 2.2% water (2.0 kg) and then the solution was gradually cooled. It was continuously cooled to 15° C. and stirred for 2.0 hours, and then cooled to −10° C. via type E crystal. Then, the crystal was separated to give Crystal form F (wet powder; 12.4 kg (purity 98.50%)) of a hemifumarate of the compound of formula (I) below:

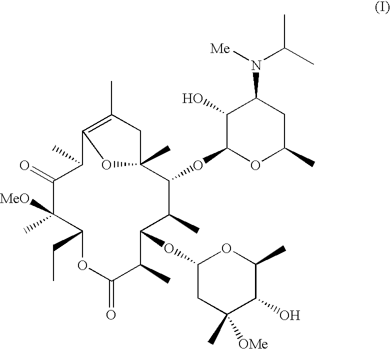

An example of the X-ray diffraction pattern of the resulting Crystal form F measured with Cu—Kα radiation is shown in FIG. 1. As apparent from FIG. 1, it showed strong peaks at diffraction angles 2θ=6.6° and 8.5°. More specifically, it showed characteristic peaks at diffraction angles 2θ=6.6°, 8.5°, 16.6°, 20.8° and 23.5°.

¹H-NMR(d₆-Acetone, ppm): 6.7 (1H, s, 1/2(═CH—COOH)₂), 5.6 (1H, dd), 4.9 (1H, d), 4.6 (1H, d), 4.1 (1H, m), 4.0-4.1 (CH₃—CH₂—O—COCH₃, q), 3.4 (3H, s), 3.2-3.3 (2H, m), 3.0 (5H, m), 2.4-2.7 (6H, m), 2.0 (CH₃—CH₂—O—COCH₃, s), 1.1 (6H, m), 0.9 (3H, t).

### [Example 2]

### Preparation of Crystal Form D Anhydrate of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)-ene-3,7-dione (E)-2-butenedioic acid salt (2:1) (Crystal Form D Anhydrate) Through Crystal Form F

The Crystal form F prepared in Example 1 (6) (12.4 kg) was dried under reduced pressure to a product temperature of 25° C. and then dried at 60° C. for 3 hours to give Crystal form D anhydrate (10.1 kg (purity 99.77%)) of a hemifumarate of the compound of formula (I) below:

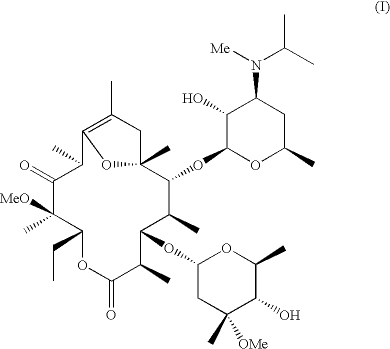

An example of the X-ray diffraction pattern of the resulting Crystal form D anhydrate measured with Cu—Kα radiation is shown in FIG. 2. As apparent from FIG. 2, it showed strong peaks at diffraction angles 2θ=7.1°, 13.5° and 14.2°. More specifically, it showed characteristic peaks at diffraction angles 2θ=7.1°, 9.4°, 10.2°, 12.3°, 13.5°, 14.2° and 16.1°. Among them, the strong peak at a diffraction angle 2θ=13.5° was a characteristic peak that was not found in Crystal form D X-hydrate described below.

m.p.: 199.2° C.

¹H-NMR (d₆-Acetone, ppm): 6.7 (1H, s, 1/2 (═CH—COOH)₂), 5.6 (1H, dd), 4.9 (1H, d), 4.6 (1H, d), 4.1 (1H, m), 3.4 (3H, s), 3.2-3.3 (2H, m), 3.0 (5H, m), 2.4-2.7 (6H, m), 1.1 (6H, m), 0.9 (3H, t).

### [Example 3]

### Preparation of Crystal Form D X-hydrate of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14 (1)-ene-3,7-dione (E)-2-butenedioic acid salt (2:1) (Crystal Form D X-hydrate) by Conditioning Crystal Form D Anhydrate

(1) Crystal form D anhydrate prepared according to the procedure of Example 2 (5.0 kg) was conditioned in an air-circulating dryer (made by Nippon Kansoki). This apparatus was fed with conditioning air at 18-20° C., 55-68% RH for 1 hour at a flow rate of 30 m³/min. As a result, 4.9 kg (purity 99.50%) of Crystal form D X-hydrate (moisture content: 2.4%) of a hemifumarate of the compound of formula (I) below was obtained.

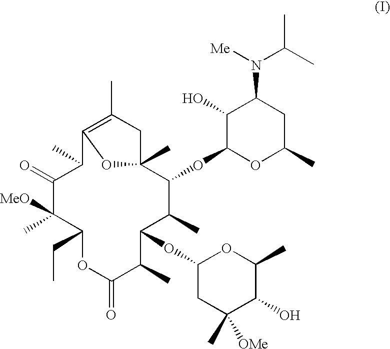

An example of the X-ray diffraction pattern of the resulting Crystal form D X-hydrate measured with Cu—Kα radiation is shown in FIG. 3. As apparent from FIG. 3, it showed strong peaks at diffraction angles 2θ=7.1° and 14.2° but did not show a strong peak at a diffraction angle 2θ=13.5° (the strong peak at a diffraction angle 2θ=13.5° found in Crystal form D anhydrate has disappeared). More specifically, it showed characteristic peaks at diffraction angles 2θ=7.1°, 10.7°, 14.2°, 15.7° and 16.7°.

This Crystal form D X-hydrate was easier to handle than Crystal form D anhydrate and therefore, advantageous in synthetic operation.

m.p.: 200.1° C.

¹H-NMR (d₆-Acetone, ppm): 6.7 (1H, s, 1/2 (═CH—COOH)₂), 5.6 (1H, dd), 4.9 (1H, d), 4.6 (1H, d), 4.1 (1H, m), 3.4 (3H, s), 3.2-3.3 (2H, m), 3.0 (5H, m), 2.4-2.7 (6H, m), 1.1 (6H, m), 0.9 (3H, t).

(2) In the same manner as described in (1) above, Crystal form D anhydrate prepared in Example 2 (9.9 kg) was conditioned in an air-circulating dryer (made by Nippon Kansoki). This apparatus was fed with conditioning air at 20-21° C., 58-63% RH for 2 hours at a flow rate of 30 m³/min. As a result, 10.1 kg (purity 99.76%) of the title compound (moisture content: 2.3%) was obtained.

The X-ray diffraction pattern measured with Cu—Kα radiation, melting point and NMR spectrum of the resulting Crystal form D X-hydrate were similar to the results shown in (1) above.

### [Example 4]

### Preparation of Crystal Form F by Placing Crystal Form D Anhydrate of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)-ene-3,7-dione (E)-2-butenedioic acid salt (2:1) in an Atmosphere Containing Aqueous Ethyl Acetate

Crystal form D anhydrate prepared in Example 1 (6) (2 g) was allowed to stand at room temperature for 15 hours in the presence of a developing layer containing 3.0% aqueous ethyl acetate (200 mL) without direct contact with the liquid. As a result, Crystal form F was obtained.

The X-ray diffraction pattern of the resulting Crystal form F measured with Cu—Kα radiation was similar to that of FIG. 1 described in Example 1 (6) above.

### [Example 5]

### Preparation of Crystal Form F by Placing Crystal Form D X-Hydrate of [2S, 4R, 5R, 8R, 9S, 10S, 11R, 12R]-9-[(2,6-dideoxy-3-C-methyl-3-O-methyl-α-L-ribo-hexopyranosyl)oxy]-5-ethyl-4-methoxy-2,4,8,10,12,14-hexamethyl-11-[[3,4,6-trideoxy-3-(isopropylmethylamino)-β-D-xylo-hexopyranosyl]oxy]-6,15-dioxabicyclo[10.2.1]pentadec-14(1)-ene-3,7-dione (E)-2-butenedioic acid salt (2:1) in an atmosphere containing aqueous ethyl acetate

Crystal form D hydrate prepared in Example 3 (2) (2 g) was allowed to stand at room temperature for 15 hours in the presence of a developing layer containing 3.0% aqueous ethyl acetate (200 mL) without direct contact with the liquid. As a result, Crystal form F was obtained.

The X-ray diffraction pattern of the resulting Crystal form F measured with Cu—Kα radiation was similar to that of FIG. 1 described in Example 1 (6) above.

### [Example 6]

### Transition Between Crystal Form D Anhydrate and Crystal Form D X-Hydrate Depending on Temperature Conditions

Crystal form D anhydrate prepared according to the procedure described in Example 2 (100 mg) was changed to Crystal form D X-hydrate by increasing the relative humidity from 0% RH to 90% RH at 25° C., and then Crystal form D X-hydrate was transferred into Crystal form D anhydrate by decreasing the relative humidity from 90% RH to 0% RH, and the moisture absorption isotherms were assessed. Measurements were made by using a dynamic moisture absorption meter DVS-1 (made by Surface Measurement Systems) at humidity points of every 2% RH within the range of 16% RH-44% RH and every 10% RH elsewhere under the conditions of a threshold dt/ds<0.002 and a threshold period=3 hrs.

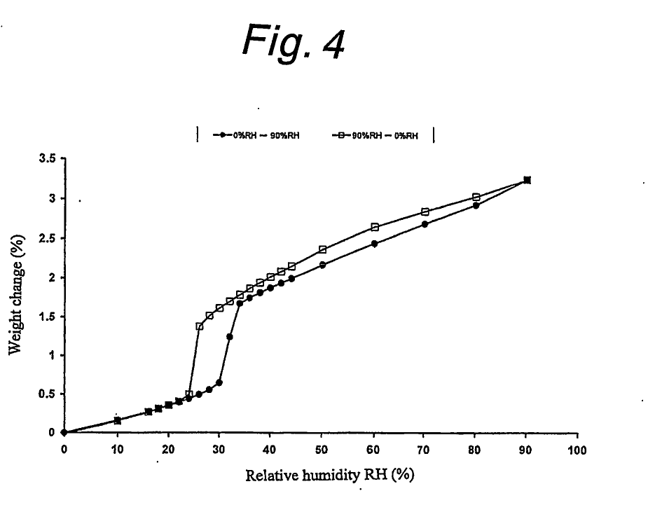

An example of the results is shown in FIG. 4. As shown in FIG. 4, a hysteresis loop was observed at 30% RH-40% RH during increasing the relative humidity from 0% RH to 90% RH and at 30% RH-20% RH during decreasing the relative humidity from 90% RH to 0% RH.

Powder X-ray diffraction analysis showed that the powder X-ray pattern changed before and after the hysteresis loop, confirming that the crystal form changed. Thus, the transition point from Crystal form D anhydrate into Crystal form D X-hydrate was situated at relative humidity 30% RH-40% RH at 25° C. and the transition point from Crystal form D X-hydrate into Crystal form D anhydrate was situated at relative humidity 30% RH-20% RH at 25° C.

Powder X-ray diffraction analysis also confirmed that the crystal form was unchanged after the hysteresis loop (40% RH or more) as shown by a constant powder X-ray pattern. Thus, the increase in moisture content after the hysteresis loop was thought to be due to the water of adhesion. If it is supposed that the increased moisture content in the hysteresis loop is crystal water, the value for X in X-hydrate was estimated at 1/2.

Each of the transition points above (hysteresis loop) tended to shift to low humidity side at temperatures lower than 25° C. and to high humidity side at temperatures higher than 25° C.

### [Example 7]

### Stability of Crystal Form D Anhydrate and Crystal Form D X-Hydrate

(1) Accelerated Testing of Crystal Form D Anhydrate and Crystal Form D X-Hydrate at 40° C. for 1 Month and at 60° C. for 1 Month

Three lots of Crystal form D anhydrate prepared according to the procedure described in Example 2 were stored and dried under reduced pressure in a desiccator containing silica gel. After drying under reduced pressure, the desiccator was charged with air conditioned at a relative humidity of 0% RH to perform accelerated testing of Crystal form D anhydrate at 40° C. for 1 month and at 60° C. for 1 month. Similarly, Crystal form D was stored in a desiccator conditioned at a relative humidity of about 75% RH by an aqueous saturated sodium chloride solution to perform accelerated testing of Crystal form D X-hydrate at 40° C. for 1 month and at 60° C. for 1 month. Before and after accelerated testing, the crystal form was confirmed to be Crystal form D anhydrate or Crystal form D X-hydrate by powder X-ray diffraction spectra.

The resulting samples were tested for purity by HPLC, and the peak area of each sample was measured by automatic analysis to determine the product percentage by the area percentage method according to the equation below. The HPLC column used was YMC ODS AM303 (4.6×250 mm) with a mobile phase consisting of a solution of acetonitrile: water=1:1 containing PICB-8 (Low UV) reagent, and a UV spectrometer (260 nm) and a PDA (200-400 nm) were used as detectors on 25 μL of each sample solution prepared by dissolving about 20 mg of each sample in a solution of acetonitrile:water=1:1 to a volume of exactly 25 mL.

Degradants percentage (%)=(degradants peak area×100)/(total of peak areas excluding peaks derived from fumaric acid and control solution)

An example of the results is shown in Table 1 below. All the lots showed that Crystal form D X-hydrate is more stable than Crystal form D anhydrate.

TABLE 1
Percent Degradants Obtained for the Crystal Form D Anhydrate and
Crystal Form D X-hydrate After Storage at Various Conditions

| | | Lot 1 | Lot 2 | Lot 3 |
|---|---|---|---|---|
| 40° C., 1 month | Anhydrate | 1.01 | 1.38 | 1.41 |
| | X-hydrate | 0.17 | 0.42 | 0.22 |
| 60° C., 1 month | Anhydrate | 2.66 | 2.52 | 3.05 |
| | X-hydrate | 0.90 | 0.63 | 1.53 |

(2) Accelerated Testing with Oxygen of Crystal Form D Anhydrate and Crystal Form D X-Hydrate at 40° C. for 2 Weeks and for 1 Month

In the same manner as described in (1) above, Crystal form D was stored and dried under reduced pressure in a desiccator containing silica gel. After drying under reduced pressure, the air in the desiccator was replaced with high-purity oxygen conditioned at a relative humidity of 0% RH to perform accelerated testing of Crystal form D anhydrate at 40° C. for 2 weeks and for 1 month. Similarly, Crystal form D was stored in a desiccator conditioned at a relative humidity of about 75% RH by an aqueous saturated sodium chloride solution and the air in the desiccator was replaced with high-purity oxygen conditioned at a relative humidity of 75% RH to perform accelerated testing of Crystal form D X-hydrate at 40° C. for 2 weeks and for 1 month. The resulting samples were analyzed as described in (1) above. Before and after accelerated testing, the crystal form was confirmed to be kept Crystal form D anhydrate or Crystal form D X-hydrate under each conditions by powder X-ray diffraction spectra.

An example of the results is shown in Table 2 below. This test also showed that Crystal form D X-hydrate is more stable than Crystal form D anhydrate.

TABLE 2
Percent Degradants Obtained for the Crystal Form D Anhydrate and
Crystal Form D X-hydrate After Storage at Various Conditions

| | | Lot 1 | Lot 2 | Lot 3 |
|---|---|---|---|---|
| Oxygen replacement | Anhydrate | 1.01 | 1.00 | 1.32 |
| 40° C., 2 weeks | X-hydrate | 0.17 | 0.05 | 0.18 |
| Oxygen replacement | Anhydrate | 3.12 | 3.39 | 3.96 |
| 40° C., 1 month | X-hydrate | 0.66 | 0.72 | 0.93 |

### [Example 8]

### Residual Solvent Levels in Crystal Form D Anhydrate and Crystal Form D X-Hydrate

Crystal form D anhydrate prepared according to Example 2 and Crystal form D X-hydrate prepared by conditioning said anhydrate according to Example 3 (1) or (2) using an air-circulating dryer (made by Nippon Kansoki), a vibro-fluidized bed dryer VUA-10DJ (made by Chuo Kakohki) or a fluidized bed granulator New Marumerizer NQ-160 (made by Fuji Paudal) were assayed for their residual solvent levels (ethyl acetate) by gas chromatography. Before and after conditioning, the crystal form was confirmed by powder X-ray diffraction spectra to have been transferred from Crystal form D anhydrate into Crystal form D X-hydrate.

An example of the results is shown in Table 3 below. In all the lots, the residual solvent levels were lower in Crystal form D X-hydrate than Crystal form D anhydrate.

TABLE 3
Residual Solvent Levels for the Crystal Form D Anhydrate and
Crystal Form D X-hydrate After Storage at Various Conditions

| | | | | Residual |
|---|---|---|---|---|
| | | | | solvent |
| | | | | level |
| | Conditioner | Conditioning conditions | | (ppm) |
| Lot A | Air-circulating | 20-21° C., 58-63% RH | Anhydrate | 697 |
| | dryer | (air) 2 hrs | (Example | |
| | | | 2) | |
| | | | X-hydrate | 636 |
| | | | (Example | |
| | | | 3(2)) | |
| Lot B | Vibro-fluidized | 22-28° C., 75 ± 15% RH | Anhydrate | 616 |
| | bed dryer | (nitrogen gas) 4 hrs | X-hydrate | 446 |
| Lot C | Fluidized bed | 24-29° C., 75 ± 10% RH | Anhydrate | 750 |
| | granulator | (air) 4 hrs | X-hydrate | 424 |
| Lot D | Vibro-fluidized | 19-26° C., 70-71% RH | Anhydrate | 875 |
| | bed dryer | (nitrogen gas) 4 hrs | X-hydrate | 424 |
| Lot E | Air-circulating | 19-28° C., 41-71% RH | Anhydrate | 663 |
| | dryer | (air) 72 hrs | X-hydrate | 251 |

A. Materials

All of the characterization described in this example was performed using the Crystal form D X-hydrate as the starting material. Solvents (either BPLC or ACS grade) and other reagents were purchased from commercial suppliers and used as received.

B. Methods

1. Solubility—Solvent Addition Method

A weighed sample of Crystal form D X-hydrate was treated with aliquots of the test solvent at room temperature. The aliquot volume was typically 100 to 500 μL. However, larger aliquot volumes were used after it was observed that the material had poor solubility in the solvent under examination. The mixture was sonicated between additions to facilitate dissolution. Complete dissolution of the test material was determined by visual inspection. Solubilities were estimated from these experiments based on the total solvent used to provide complete dissolution. The actual solubilities may be greater than those calculated because of the use of solvent aliquots that were too large or due to a slow rate of dissolution. The solubility is expressed as “less than” if dissolution did not occur during the experiment. If complete dissolution was achieved as a result of only one aliquot addition, the solubility is expressed as “greater than.”

2. Polymorph Screen

A polymorph screen was carried out to identify as many solid forms as possible. Both thermodynamic (evaporation, slurry and slow cool) and linetic (antisolvent precipitation and crash cool precipitation) crystallization techniques were employed. These techniques are described in more detail below. Once solid samples were harvested from crystallization attempts, they were examined under a microscope for birefringence and morphology. Any crystalline shape was noted, but sometimes the solid exhibited unknown morphology, due to small particle size. Solid samples were then analyzed by XRPD, and the crystalline patterns compared to each other to identify new crystalline forms.

a. Fast Evaporation (FE)

Solutions of the Crystal form D X-hydrate crystal were prepared in various solvents and sonicated between aliquot additions to assist in dissolution. Once a mixture reached complete dissolution, as judged by visual observation, the solution was filtered through a 0.2-μm nylon filter. The filtered solution was allowed to evaporate at a specific temperature (typically ambient) in an open vial. The solids that formed were isolated and analyzed.

b. Slow Evaporation (SE)

Solutions of the Crystal form D X-hydrate crystal were prepared in various solvents and sonicated between aliquot additions to assist in dissolution. Once a mixture reached complete dissolution, as judged by visual observation, the solution was filtered through a 0.2-μm nylon filter. The filtered solution was allowed to evaporate at a specific temperature (typically ambient) in a vial covered with aluminum foil perforated with pinholes. The solids that formed were isolated and analyzed.

C. Slow Cool (SC)

Saturated solutions of the Crystal form D X-hydrate crystal were prepared in various solvents at an elevated temperature of 60° C. and filtered through a 0.2-μm nylon filter into an open vial while still warm. The vial was covered and allowed to cool slowly to room temperature. The presence or absence of solids was noted. If there were no solids present, or if the amount of solids was judged too small for XRPD analysis, the vial was placed in a refrigerator overnight. Again, the presence or absence of solids was noted and if there were none, the vial was placed in a freezer overnight. Solids that formed were isolated by filtration and allowed to dry prior to analysis.

d. Crash Cool (CC)

Saturated solutions of the Crystal form D X-hydrate crystal were prepared in various solvents or solvent systems at an elevated temperature of 60° C. and filtered through a 0.2-μm nylon filter into an open vial while still warm. The vial was covered and placed directly into a freezer. The presence or absence of solids was noted. Solids that formed were isolated by filtration and allowed to dry prior to analysis.

e. Rotary Evaporation

Solutions of the Crystal form D X-hydrate crystal were prepared in various solvents and filtered through a 0.2-μm nylon filter. The sample was placed on the rotary evaporator and removed when dry. Typically, slow cool or crash cool experiments that did not produce solids were used for rotary evaporation experiments.

f. Crash Precipitation (CP)

Solutions of the Crystal form D X-hydrate crystal were prepared in various solvents and filtered through a 0.2-μm nylon filter. Solid formation was induced by adding the filtered solution to an appropriate anti-solvent at a given temperature. The resulting solids, were isolated by filtration and dried prior to analysis. In cases where no solids formed immediately, the samples were placed in a freezer to facilitate crystallization.

g. Slurry Experiments

Solutions of the Crystal form D X-hydrate crystal were prepared by adding enough solids, to a given solvent so that undissolved solids were present. The mixture was then agitated in a sealed vial at a given temperature. After several days, the solids were isolated by suction filtration.

C. Instrumental Techniques

1. Coulometric Kart Fischer Analyses

Coulometric Karl Fischer (KF) analysis for water determination was performed using a Mettler Toledo DL39 Karl Fischer filtrator. Approximately 50 mg of sample was placed in the KF titration vessel containing approximately 100 mL of Hydranal-Coulomat AD and mixed for 60 seconds to ensure dissolution. The sample was then titrated by means of a generator electrode which produces iodine by electrochemical oxidation: 2 I−=>I₂+2e. Three replicates were obtained to ensure reproducibility.

2. Differential Scanning Calorimetry (D)SC)

Analyses were carried out on a TA Instruments differential scanning calorimeter 2920. The instrument was calibrated using indium as the reference material. The sample was placed into an aluminum DSC pan, and the weight accurately recorded. Two pan configurations were utilized. The pan was either covered with a lid perforated with a laser pinhole to allow for pressure release, and then hermetically scaled; or open with no lid. Each sample was equilibrated at 25° C. and heated under a nitrogen purge at a rate of 10° C./min.

3. Hot Stage Optical Microscopy

Hot stage optical microscopy was carried out using a Kofler hot stage mounted on a Leica DM LP microscope equipped with a Sony DXC-970MD 3CM camera and Linksys version 2.27 for collecting images. The solid material was placed onto a glass slide and covered with a cover slip. The sample was visually observed at an objective of 20× as the stage was heated and observations noted. The hot stage was temperature calibrated using USP standards vanillin and caffeine.

4. IR Spectra

Infrared spectra were acquired on a Magna-IR 860® Fourier transform infrared (FT-IR) spectrophotometer (Thermo Nicolet) equipped with an Ever-Glo mid/far IR source, a potassium bromide (KBr) beamsplitter, and a deuterated triglycine sulfate (DTGS) detector. A diffuse reflectance accessory (the Collector™, Thermo Spectra-Tech) was used for sampling. Each spectrum represents 256 co-added scans collected at a spectral resolution of 4 cm⁻¹. Sample preparation consisted of placing the sample into a 13-mm diameter cup and leveling the material with a frosted glass slide. A background data set was acquired with an alignment mirror in place. A Log 1/R (R=reflectance) spectrum was acquired by taking a ratio of these two data sets against each other. Wavelength calibration was performed using polystyrene.

5. Mass Spectrometry

Mass spectrometry was carried out at M-Scan Inc., West Chester, Pa. The analyses were performed using a ZAB 2-SE high field mass spectrometer. A cesium ion gun was used to generate ions for the acquired mass spectra, which were recorded using a PDP 11-250J data system. Mass calibration was performed using cesium iodide.

6. Moisture Sorption/Desorption Analysis

Data were collected on a VTI SGA-100 moisture balance system. For sorption isotherms, a sorption range of 5 to 95% relative humidity (RH) and a desorption range of 95 to 5% RH in 10% RH increments were used for analysis. The samples were not dried prior to analysis. Equilibrium criteria used for analysis were less than 0.0100% weight change in 5 minutes with a maximum equilibration time of 3 hours if the weight criterion was not met. Data were not corrected for the initial moisture content of the samples.

7. Nuclear Magnetic Resonance (NMR)

The solution phase ¹H NMR spectra were acquired at ambient temperature on a Bruker Instrument model AM 250 spectrometer operating at 5.87 T (Lannor frequency: ¹H=250 MHz). Samples were dissolved in NMR-grade acctonitrile-d₃ or chloroform-d. Data was collected with a 4.0-μs or 7.5-μs pulse width, a spectral width of 5000 Hz, and a relaxation delay time of 5.00 s. Each ¹H NMR spectrum represents 128 co-added transients. The free induction decay (FID) was exponentially multiplied with a 0.1 Hz Lorentzian line broadening factor to improve the signal-to-noise ratio. Spectra were processed utilizing GRAMS/32 AI version 6.00. Predicted chemical shift values were calculated using ChemDraw Pro version 4.5 [1].

8. Optical Microscopy

Optical microscopy data was collected on a Wolfe polarizing optical microscope at a magnification of 20×. Crossed polarizers were used to observe birefringence in the samples.

9. Raman Spectra

Raman spectra were acquired on either a Nicolet FT-Raman 960 spectrometer or a Raman accessory module interfaced to a Magna 860® Fourier transform infrared (FI-IR) spectrophotometer (Thermo Nicolet). Both spectrometers use an excitation wavelength of 1064 nm. Approximately 0.5 W (variable) of laser power was used to irradiate the samples. The Raman spectra were measured with an indium gallium arsenide (InGaAs) detector. The samples were prepared for analysis by placing them in an NMR tube. The NMR tube was placed in a gold-coated NMR tube holder. Each spectrum is the result of 256 co-added scans acquired at 4 cm⁻¹ resolution and an autogain setting. The spectrometer was calibrated (wavelength) with sulfur and cyclohexane at the time of use.

10. Thermogravimetric Analysis (TGA)

Analyses were carried out on either TA Instruments 2050 or 2950 thermogravimetric analyzer. The calibration standards were nickel and Alumel™. Each sample was placed in an aluminum sample pan and inserted into the TG furnace. Samples were first equilibrated at 25 ′C, then heated under a stream of nitrogen at a heating rate of 10 ′Clmin.

11. Thermogravimetric Infrared Analysis (TG-IR)

Thermogravimetric infrared (TGAR) analyses were acquired on a TA Instruments thermogravimetric (TG) analyzer model 2050 interfaced to a Magna 560® Fourier transform infrared (FT-IR) spectrophotometer (Thermo Nicolet) equipped with a Ever-Glo mid/far IR source, a potassium bromide (KBr) beamsplitter, and a deuterated triglycine sulfate (DTGS) detector. The TG instrument was operated under a flow of helium at 90 and 10 cc/min for the purge and balance, respectively. Each sample was placed in a platinum sample pan, inserted into the TG furnace, accurately weighed by the instrument, and heated from ambient at a rate of 20° C./min. The TG instrument was started first, immediately followed by the FT-IR instrument. Each IR spectrum represents 32 co-added scans collected at a spectral resolution of 8 cm⁻¹. IR spectra were collected every 18 seconds. A background scan was collected before the beginning of the experiment. Wavelength calibration was performed using polystyrene. The TG calibration standards were nickel and Alumel™. Volatiles were identified

### [Example 9]

### Preparation of Crystal form G1

Iso-Structural Crystal Form G1.

Hydrate D-form crystal in an amount of 0.200 g was dissolved in 5.0 ml acetone, assisted with sonication, at ambient. The clear solution was filtered through a 0.2-um nylon filter. The filtrate was evaporated to dryness (1 day) in an open container at ambient temperature to allow the product to crystallize to give the hemifumarate salt as a variable hydrate-solvate.

### [Example 10]

### Preparation of Crystal form G2

Iso-Structural Crystal Form G2.

Hydrate D-form crystal in an amount of 0.028 g was dissolved in 2.1 ml of methylethylketone, assisted with sonication, at ambient. The clear solution was filtered through a 0.2-um nylon filter. The filtrate was evaporated to dryness (4 days) in an open container at ambient temperature to allow the product to crystallize to give the hemifumarate salt as a variable hydrate-solvate.

### [Example 11]

### Preparation of Crystal form G3

Iso-Structural Crystal Form G3.

Hydrate D-form crystal in an amount of 0.031 g was dissolved in 0.6 ml of tetrahydrofuran, assisted with sonication, at ambient. The clear solution was filtered through a 0.2-um nylon filter. The filtrate was evaporated to dryness (4 days) in an open container at ambient temperature to allow the product to crystallize to give the hemifumarate salt as a variable hydrate-solvate.

### [Example 12]

### Characterization of Iso-Structural Solvate Crystal Form G1, Crystal Form G2 and Crystal Form G3

The following experimental methods were adopted in order to characterize and identify each of Crystal form G1, Crystal form G2 and Crystal form G3 crystals.

a. Crystal Form G1

Crystal form G1 was obtained from evaporative experiments with acetone. A representative XRPD (X-ray powder diffraction) pattern is shown in FIG. 5. The XRPD pattern of Crystal form G1 is nearly identical to Crystal form G2 (methylethylketone) and Crystal form G3 (tetrahydrofuran). This may indicate that G0-G3-form materials are isostructural solvates. Additional characterization data obtained on Crystal form G2 and Crystal form G3 is provided in sections b and c below.

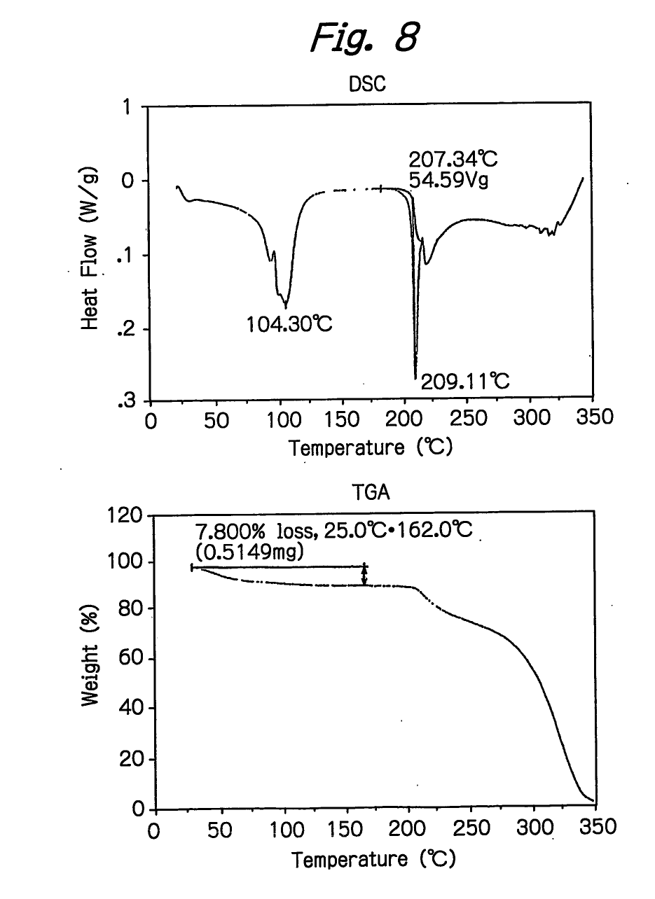

Thermal data for Crystal form G1 is plotted in FIG. 8. The DSC (differential scanning calorimetry) curve exhibited multiple broad endothermic events occurring around 104° C. and an additional endothermic event exhibiting an onset temperature of 207° C. The nature of these events was not confirmed by hot stage microscopy.

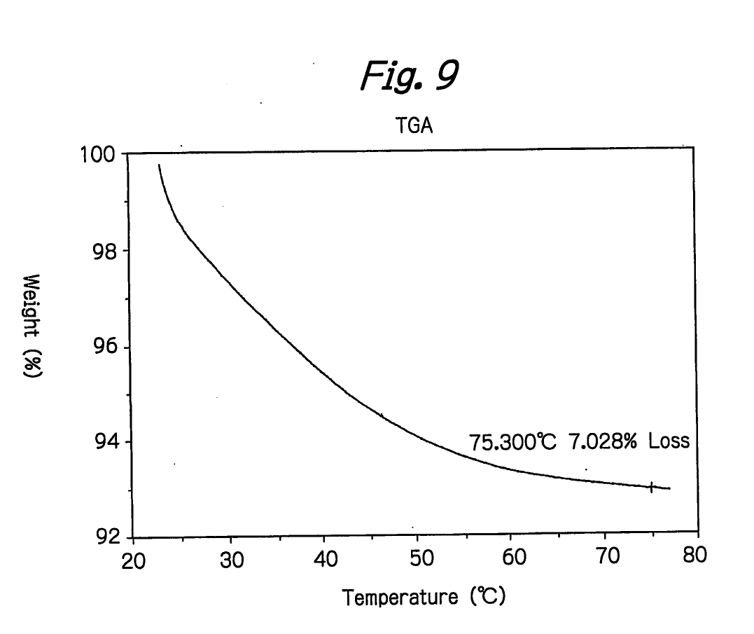

The TG (thermogravimetric) curve obtained on Crystal form G1 material shows a weight loss of approximately 7.8% between 26° C. and 162° C. The total weight loss corresponds to approximately 1.2 moles of acetone. A separate TG experiment (FIG. 9) was done in order to determine if another form could be acquired through desolvation. Crystal form G1 was heated to 75° C., providing a weight loss of approximately 7.0%. Weight loss in the TG data is observed to occur at or near the beginning of the experiment, indicating that some volatilization occurs under these conditions (dry helium flow). Based on these data, the TG weight loss may not provide an accurate measure of the solvation state of this material. Upon cooling to room temperature, this material was recovered and an XRPD pattern obtained. This pattern indicated conversion of Crystal form G1 to a low crystalline or amorphous pattern. Insufficient material was available to complete attempts to obtain TG-IR (thermogravimetric infrared analysis) data on this material in order to identify the volatile component.

Based on the characterization data, Crystal form G1 appears to be a crystalline, acetone solvate which is iso-structural with Crystal form G2 and Crystal form G3. Low crystalline or amorphous material was acquired through attempts to desolvate this material at an elevated temperature. However, based on the characterization of the other two G-form materials, Crystal form GO may be a mixed solvate-hydrate material.

b. Crystal Form G2

Crystal form G2 was obtained from evaporative experiments with methylethylketone. A representative XRPD pattern is shown in FIG. 6. The XRPD pattern of Crystal form G2 is nearly identical to Crystal form G1 and Crystal form G3. This indicates that Crystal form G2 is likely an iso-structural solvate of Crystal form G1 and Crystal form G3 (refer to sections a above and c below).

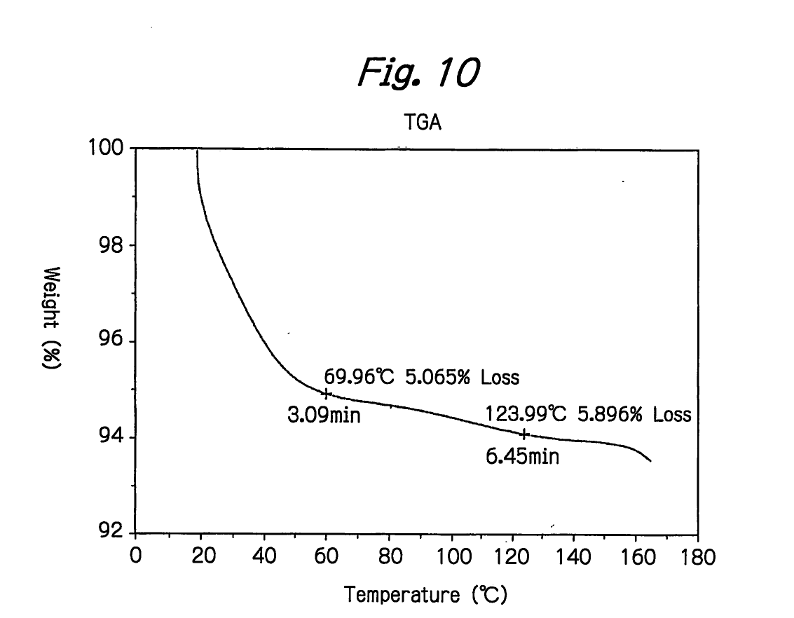

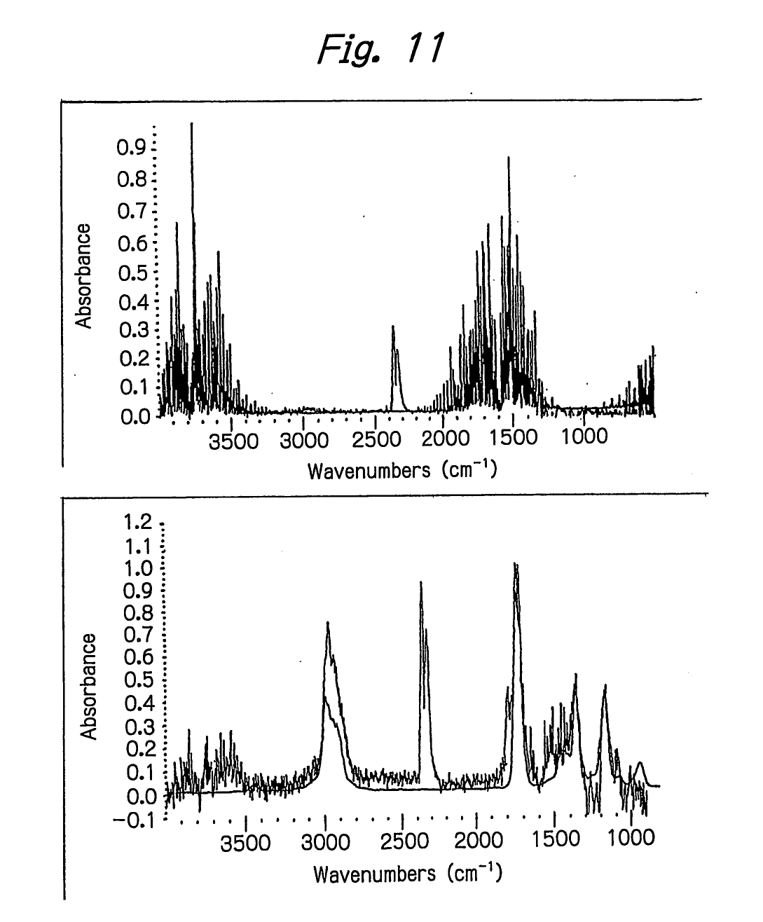

A TG-IR experiment was done to determine the nature of the solvate and if another form could be acquired through its desolvation. Weight loss in the TG data (FIG. 10) is observed to occur at or near the beginning of the experiment, indicating that some volatilization occurs under these conditions (dry helium flow). Based on these data, the TG weight loss may not provide an accurate measure of the solvation state of this material. Upon cooling to room temperature, this material was recovered and an XRPD pattern obtained. This pattern indicated conversion of Crystal Form G2 material to Crystal form F. Crystal form F is an anhydrous, crystalline material. The IR spectra identify water and methylethylketone as the volatiles removed during desolvation from ambient up to 60° C. and methylethylketone as the volatile subsequently following (FIG. 11). This indicates that Crystal form G2 is a mixed hydrate-solvate.

Based on the characterization data, Crystal form G2 appears to be a crystalline, mixed hydrate-methylethylketone solvate which is iso-structural with forms Crystal form G1 and Crystal form G3.

C. Crystal Form G3

Crystal form G3 was obtained from evaporative experiments with tetrahydrofuran. A representative XRPD pattern is shown in FIG. 7. The XRPD pattern of Crystal form G3 is nearly identical to Crystal form G1 and Crystal form G2. This indicates that Crystal form G3 is likely an iso-structural solvate of Crystal form G1 and Crystal form G2, obtained from acetone and methylethylketone, respectively (refer to sections a and b above).

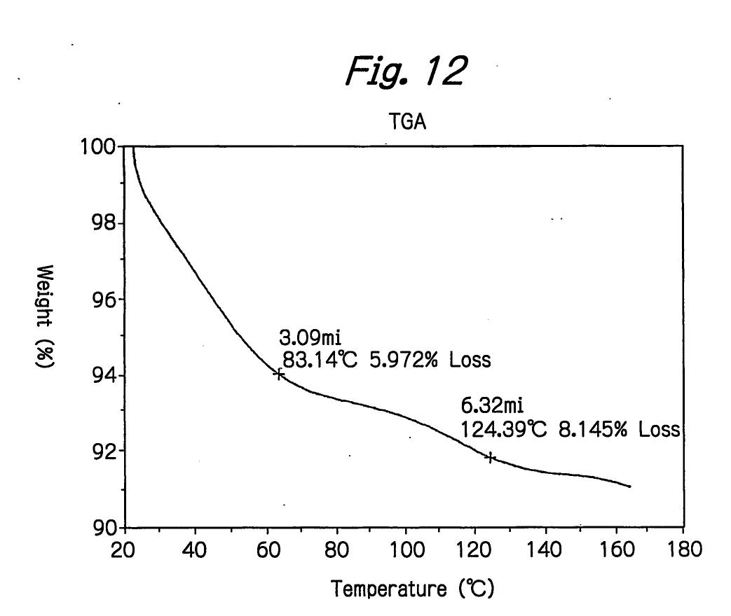

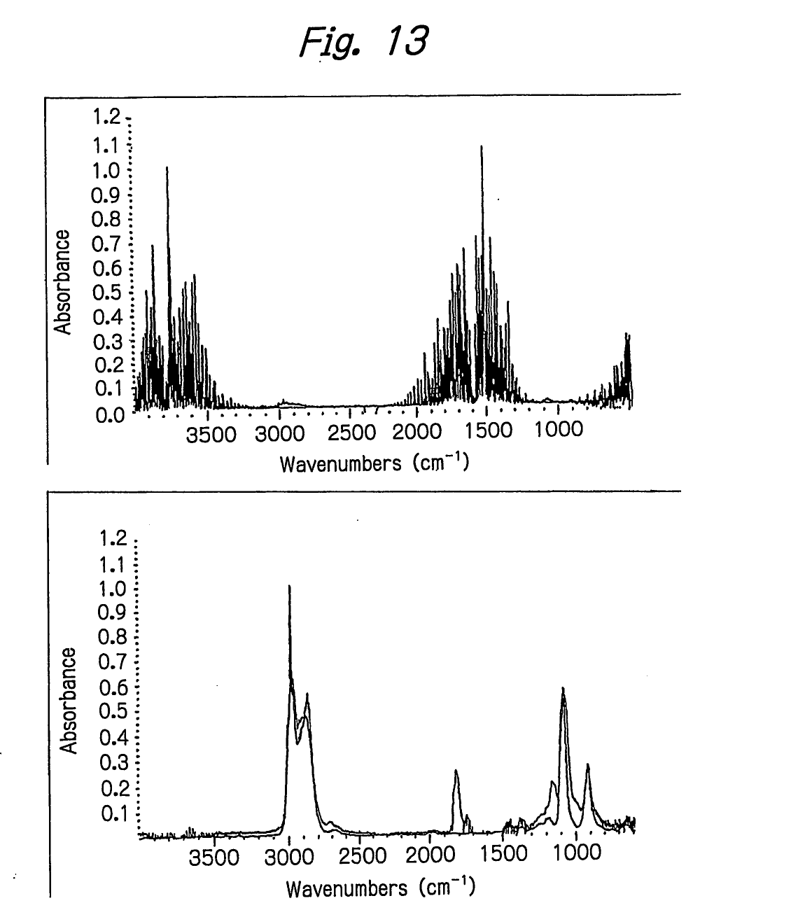

A TG-IR experiment was done to determine the nature of the solvate and if another form could be acquired through its desolvation. Weight loss in the TG data (FIG. 12) is observed to occur at or near the beginning of the experiment, indicating that some volatilization occurs under these conditions (dry helium flow). Based on these data, the TG weight loss may not provide an accurate measure of the solvation state of this material. Upon cooling to room temperature, this material was recovered and an XRPD pattern obtained. This pattern indicated conversion of Crystal form G3 material to a low crystalline or amorphous pattern. The IR spectra identify water and tetrahydrofuran as the volatiles removed during desolvation from ambient up to 63° C. and tetrahydrofuran as the volatile subsequently following (FIG. 13). This indicates that Crystal form G3 is a mixed hydrate-solvate.

Based on the characterization data, Crystal form G3 appears to be a crystalline, hydrate-tetrahydrofuran solvate which is iso-structural with forms Crystal form G1 and Crystal form G2.

### [Example 13]

### XRPD Characterization of G-Form Crystal

Table 4 contains XRPD line positions of Form G. All four lines are required to be present, since the individual lines are observed in other forms. Line positions are rounded to the nearest 0.1° 2θ and reported as ±0.2° 2θ. Table 5 contains the all of the experimental line positions for the same sample with relative intensities (I/Io) greater than 10 and in the range of 4 to 40°2θ.

TABLE 4
Experimental XRPD Line Positions for Form G

| Peak No. | 2Theta^{a} |
|---|---|
| 1 | 5.4 |
| 2 | 10.4 |
| 3 | 10.7 |
| 4 | 12.1 |

^{a}Line positions rounded to the nearest 0.1° 2θ and reported as ±0.2° 2θ.

TABLE 5
Experimental XRPD Peak List for Form G1 with
Relative Initensity (I/Io) Greater than 10

| Peak No. | 2Theta^{a} | I/Io^{b} |
|---|---|---|
| 1 | 4.2 | 30 |
| 2 | 4.5 | 28 |
| 3 | 4.7 | 30 |
| 4 | 4.9 | 34 |
| 5 | 5.4 | 68 |
| 6 | 5.9 | 24 |
| 7 | 6.1 | 21 |
| 8 | 6.5 | 24 |
| 9 | 6.9 | 21 |
| 10 | 7.3 | 20 |
| 11 | 7.5 | 19 |
| 12 | 7.9 | 23 |
| 13 | 8.2 | 18 |
| 14 | 8.4 | 18 |
| 15 | 8.7 | 17 |
| 16 | 9.0 | 19 |
| 17 | 9.2 | 19 |
| 18 | 9.4 | 19 |
| 19 | 9.8 | 37 |
| 20 | 10.1 | 38 |
| 21 | 10.4 | 72 |
| 22 | 10.7 | 100 |
| 23 | 11.1 | 34 |
| 24 | 11.7 | 33 |
| 25 | 12.1 | 75 |
| 26 | 12.5 | 34 |
| 27 | 12.9 | 24 |
| 28 | 13.2 | 19 |
| 29 | 13.5 | 22 |
| 31 | 14.3 | 15 |
| 33 | 15.1 | 12 |
| 36 | 16.2 | 13 |
| 41 | 17.9 | 29 |
| 42 | 18.5 | 13 |
| 45 | 19.7 | 24 |
| 47 | 20.5 | 12 |

^{a}XRPD line position rounded to the nearest 0.1° 2θ and reported as ±0.2° 2θ.
^{b}Experimental relative intensities for comparison purposes only.

### [Example 15]

### Utility of G Forms

Crystal Form D Via Iso-Structural Crystal Form G2

Iso-structural Crystal form G2 crystal in an amount of 0.010 g was heated from ambient temperature to 165° C. under a constant purge of dry helium. The product was allowed to cool to ambient to give the hemifumarate salt as the anhydrate Crystal form D.

### INDUSTRIAL APPLICABILITY

Crystal form D X-hydrate of the present invention is especially useful as a pharmaceutical material because of the low residual solvent level, high stability even as a compound and easy handling. Crystal form D anhydrate of the present invention is useful as a material or an intermediate for the synthesis of this Crystal form D X-hydrate. Crystal form F of the present invention is useful as a material or an intermediate for the synthesis of these Crystal form D anhydrate and Crystal form D X-hydrate. The process for preparing Crystal form D X-hydrate by conditioning Crystal form D anhydrate according to the present invention is an inexpensive and simple process that can provide Crystal form D X-hydrate having stable quality.

1. A hemifumarate crystal of a compound of formula (I): 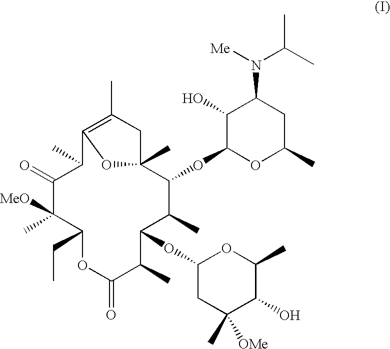
characterized by 2-theta angle positions in the powder X-ray diffraction pattern of 6.6° and 8.5°.

2. A hemifumarate anhydrate of a compound of formula (I): 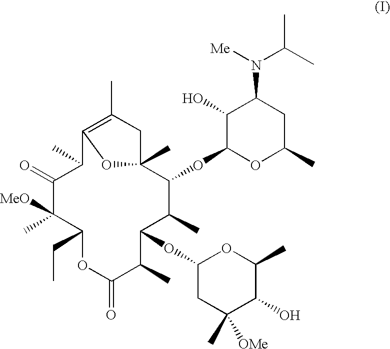
characterized by 2-theta angle positions in the powder X-ray diffraction pattern of 7.1°, 13.5° and 14.2°.

3. A hemifumarate X-hydrate of a compound of formula (I): 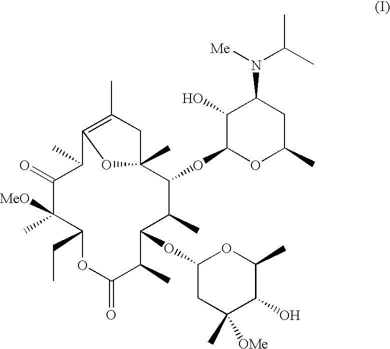
characterized by 2-theta angle positions in the powder X-ray diffraction pattern of 7.1° and 14.2°.

4. A process for preparing a hemifumarate X-hydrate of a compound of formula (I): 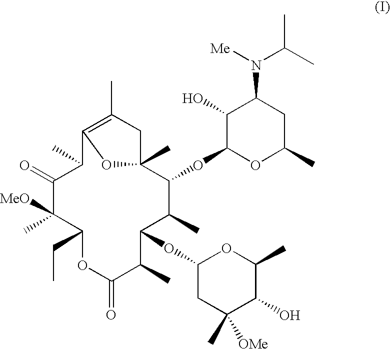
characterized by 2-theta angle positions in the powder X-ray diffraction pattern of 7.1°, said process comprising the step of treating a hemifumarate anhydrate of the compound of formula (I) characterized by 2-theta angle positions in the powder X-ray diffraction pattern of 7.1°, 13.5° and 14.2°, to obtain said hemifumarate X-hydrate.

5. A process for preparing a hemifumarate anhydrate of a compound of formula (I): 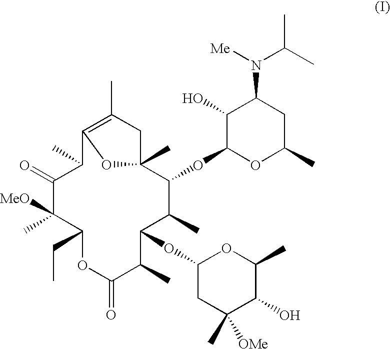
characterized by 2-theta angle positions in the powder X-ray diffraction pattern of 7.1°, 13.5° and 14.2°, said process comprising the step of treating a hemifumarate crystal form of the compound of formula (I) characterized by 2-theta angle positions in the powder X-ray diffraction pattern of 6.6° and 8.5°, to obtain said hydrate.

6. A process for preparing a hemifumarate X-hydrate of a compound of formula (I): 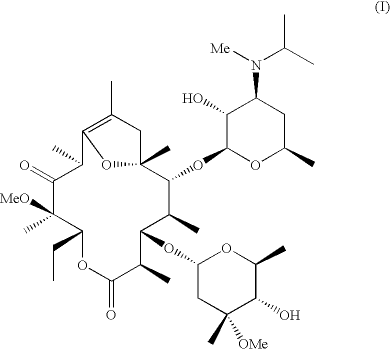
characterized by 2-theta angle positions in the powder X-ray diffraction pattern of 7.1° and 14.2°, said process comprising the step of treating a hemifumarate crystal of the compound of formula (I) 6.6° and 8.5°, to obtain said hydrate.

7. A process for preparing a hemifumarate X-hydrate of a compound of formula (I): 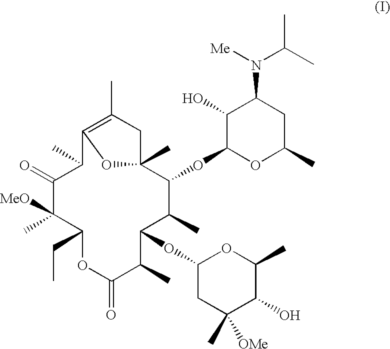
characterized by 2-theta angle positions in the powder X-ray diffraction pattern of 7.1° and 14.2°, said process comprising the step of treating a hemifumarate anhydrate of the compound of formula (I) characterized by 2-theta angle positions in the powder X-ray diffraction pattern of 7.1°, 13.5° and 14.2°, wherein said hemifumarate anhydrate is obtained by treating a hemifumarate crystal of the compound of formula (I) characterized by 2-theta angle positions in the powder X-ray diffraction pattern of 6.6° and 8.5°.

8. A hemifumarate crystal of a compound of formula (I): 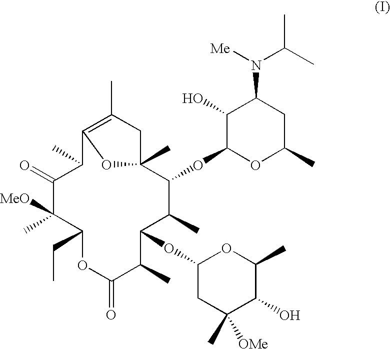
characterized by 2-theta angle positions in the powder X-ray diffraction pattern of 5.4°, 10.4°, 10.7° and 12.1°.

9. A hemifumarate crystal of a compound of formula (I): 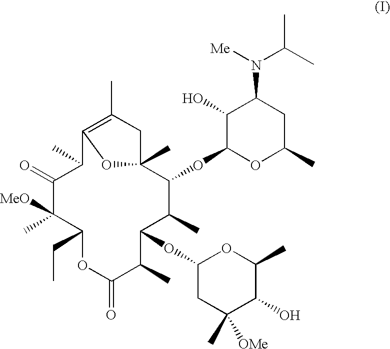
containing acetone and showing strong X-ray diffraction peaks at diffraction angles 2 theta=5.4°, 10.4°, 10.7° and 12.1° measured by X-ray diffractometry using Cu—Kα radiation.

10. A hemifumarate crystal of a compound of formula (I): 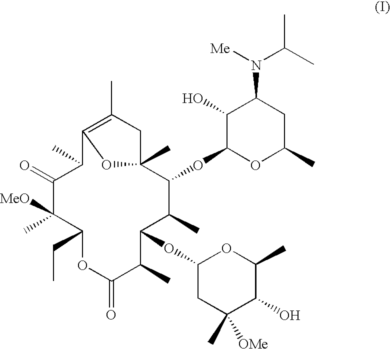
containing methylethylketone and showing strong X-ray diffraction peaks at diffraction angles 2 theta=5.4°, 10.4°, 10.7° and 12.1° measured by X-ray diffractometry using Cu—Kα radiation

11. A hemifumarate crystal of a compound of formula (I): 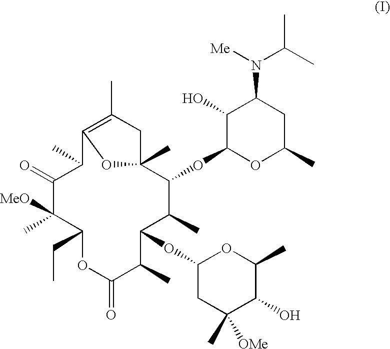
containing tetrahydrofuran and showing strong X-ray diffraction peaks at diffraction angles 2 theta=5.4°, 10.4°, 10.7° and 12.1° measured by X-ray diffractometry using Cu—Kα radiation.

12. A process for preparing a hemifumarate X-hydrate of a compound of formula (I): 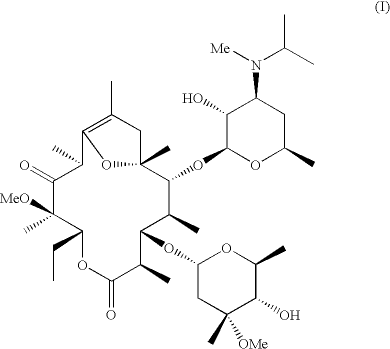
showing strong X-ray diffraction peaks at diffraction angles 2 theta=5.4°, 10.4°, 10.7° and 12.1° measured by X-ray diffractometry using Cu—Kα radiation, said process comprising the step of treating a hemifumarate crystal of the compound of formula (I) characterized by 2-theta angle positions in the powder X-ray diffraction pattern of 5.4°, 10.4°, 10.7° and 12.1°, to obtain said hydrate.

13. A process for preparing a hemifumarate X-hydrate of a compound of formula (I): 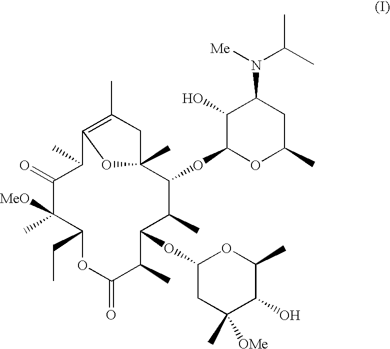
containing acetone and showing strong X-ray diffraction peaks at diffraction angles 2 theta=5.4°, 10.4°, 10.7° and 12.1° measured by X-ray diffractometry using Cu—Kα radiation, said process comprising the step of treating a hemifumarate crystal of the compound of formula (I) characterized by 2-theta angle positions in the powder X-ray diffraction pattern of 5.4°, 10.4°, 10.7° and 12.1°, to obtain said hydrate.

14. A process for preparing a hemifumarate X-hydrate of a compound of formula (I): 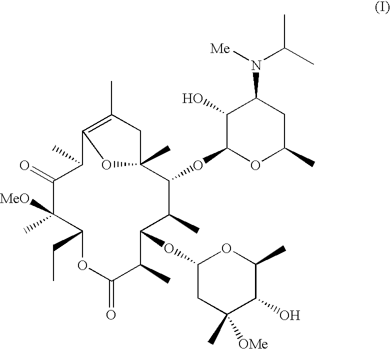
containing methylethylketone and showing strong X-ray diffraction peaks at diffraction angles 2 theta=5.4°, 10.4°, 10.7° and 12.1° measured by X-ray diffractometry using Cu—Kα radiation, said process comprising the step of treating a hemifumarate crystal of the compound of formula (I) characterized by 2-theta angle positions in the powder X-ray diffraction pattern of 5.4°, 10.4°, 10.7° and 12.1°, to obtain said hydrate.

15. A process for preparing a hemifumarate X-hydrate of a compound of formula (I): 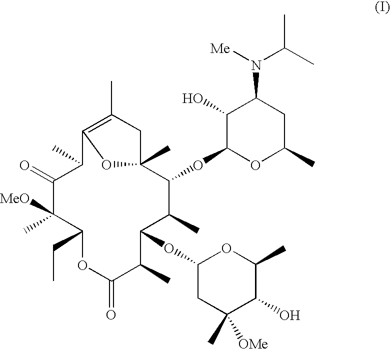
containing tetrahydrofuran and showing strong X-ray diffraction peaks at diffraction angles 2 theta=5.4°, 10.4°, 10.7° and 12.1° measured by X-ray diffractometry using Cu—Kα radiation, said process comprising the step of treating a hemifumarate crystal of the compound of formula (I) characterized by 2-theta angle positions in the powder X-ray diffraction pattern of 5.4°, 10.4°, 10.7° and 12.1°, to obtain said hydrate.

16. A process for preparing a hemifumarate anhydrate of a compound of formula (I): 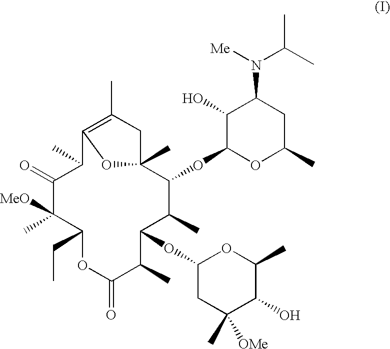
characterized by 2-theta angle positions in the powder X-ray diffraction pattern of 7.1°, 13.5° and 14.2°, said process comprising the step of obtaining said anhydrate by treating a hemifumarate crystal of claim 8, 9, 10 or 11.

17. A process for preparing a hemifumarate X-hydrate of a compound of formula (I): 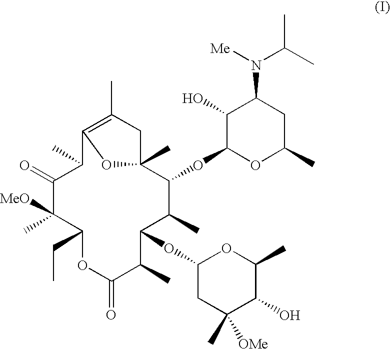
characterized by 2-theta angle positions in the powder X-ray diffraction pattern of showing strong X-ray diffraction peaks at diffraction angles 2θ=7.1° and 14.2°, said process comprising the step of obtaining said hydrate by treating a hemifumarate crystal of claim 8, 9, 10 or 11.

18. A process for preparing a hemifumarate X-hydrate of a compound of formula (I): 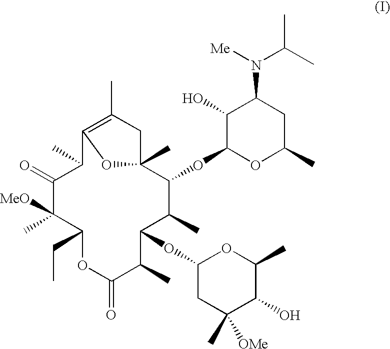
characterized by 2-theta angle positions in the powder X-ray diffraction pattern of 7.1° and 14.2°, said process comprising the step of treating a hemifumarate anhydrate of the compound of formula (I) characterized by 2-theta angle positions in the powder X-ray diffraction pattern of 7.1°, 13.5° and 14.2°, wherein said anhydrate is obtained by treating a hemifumarate crystal of claim 8, 9, 10 or 11.
